(19) United States
(12) Patent Application Publication
(10) Pub. No.: US 2026/0131073 A1
(43) Pub. Date: May 14, 2026

(54) INTEGRATED CGM AND INSULIN PUMP SYSTEM WITH DIRECT POWER CONNECTION

(71) Applicant: Tandem Diabetes Care, Inc, San Diego, CA (US)

(72) Inventors: Reza Farshchi, San Diego, CA (US); Michael Michaud, San Diego, CA (US)

(21) Appl. No.: 19/379,377

(22) Filed: Nov. 4, 2025

Related U.S. Application Data
(60) Provisional application No. 63/719,453, filed on Nov. 12, 2024.

(51) Int. Cl. A61M 5/142; A61M 5/172

(52) U.S. Cl. CPC A61M 5/14244; A61M 2005/1726; A61M 2230/201

(57) ABSTRACT

Ambulatory infusion pump systems can integrate a CGM sensor with an ambulatory infusion pump via a pump holder or tray that releasably holds the pump and is configured to be worn by a user. A direct electrical connection between the pump and the CGM can be facilitated by providing a sensor nest within the pump holder that causes an electrical connector on the pump to directly interface with the sensor. By combining the CGM and insulin pump into one integrated system with a direct electrical connection, a more aesthetically pleasing, convenient and reliable system is provided. Diabetes management is simplified by consolidating two essential devices into one.

### PRIORITY CLAIM

The present application claims the benefit of U.S. Provisional Application No. 63/719,453 filed Nov. 12, 2024, which is hereby incorporated herein by reference in its entirety.

### TECHNICAL FIELD

The present disclosure relates generally to ambulatory infusion pumps and, more particularly, to operation of ambulatory infusion pumps in conjunction with a continuous glucose monitor.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with Type 1 or Type 2 diabetes. Some insulin injecting pumps are configured as portable or ambulatory infusion devices that can provide continuous subcutaneous insulin injection and/or infusion therapy as an alternative to multiple daily injections of insulin via a syringe or an insulin pen. Such pumps can be worn or carried by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Ambulatory infusion pumps for delivering insulin or other medicaments can be used in conjunction with blood glucose monitoring systems, such as continuous glucose monitoring devices (CGMs). A CGM device may consist of a sensor placed under the patient's skin and affixed to the patient via an adhesive patch, a transmitter, and a monitor. A CGM device samples the patient's interstitial fluid periodically (e.g., once every 1-5 minutes) to estimate blood glucose levels over time. CGMs are advantageous because they provide more frequent insights into a user's blood glucose levels yet do not require a finger stick each time a reading is taken.

Ambulatory infusion pumps may communicate with a dedicated CGM directly via a wired connection or indirectly via a wireless connection using wireless data communication protocols to communicate with a separate device (e.g., a dedicated remote device or a smartphone). One example of integration of ambulatory infusion pumps with CGM devices is described in U.S. Patent Publication No. 2014/0276419, which is hereby incorporated by reference herein. Ambulatory infusion pumps typically allow the user or caregiver to adjust the amount of insulin or other medicament delivered by a basal rate or a bolus, based on blood glucose data obtained by a CGM device, and in some cases include the capability to automatically adjust such medicament delivery. For example, based on CGM readings, some ambulatory infusion pumps may automatically adjust or prompt the user to adjust the level of medicament being administered or planned for administration or, in cases of abnormally low blood glucose readings, reducing or temporarily ceasing insulin administration.

While integrations of CGM devices with insulin pumps provides a number of advantages, the CGM sensor and pump are separately attached to the body resulting in a cumbersome and visually unappealing dual device configuration. Both the pump and the CGM also need to each have a battery, processor, communications element, etc. In addition, in order to establish wireless communications between the devices a pairing procedure must be undertaken. Patients are therefore burdened with managing two distinct systems, each with its own power source and attachment method, leading to increased complexity, higher costs and a less streamlined user experience.

### SUMMARY

Embodiments disclosed herein are directed to ambulatory infusion pump systems that integrate a CGM sensor with an ambulatory infusion pump via a pump holder or tray that releasably holds the pump and is configured to be worn by a user. A direct electrical connection between the pump and the CGM can be facilitated by providing a sensor nest within the pump holder that causes an electrical connector on the pump to directly interface with the sensor. By combining the CGM and insulin pump into one integrated system with a direct electrical connection, a more aesthetically pleasing, convenient and reliable system is provided. Diabetes management is simplified by consolidating two essential devices into one.

In embodiments, an ambulatory infusion pump system can include an ambulatory infusion pump including a housing, a reservoir configured to contain a medicament, a pumping mechanism configured to deliver the medicament from the reservoir to a user, a battery and an electrical connector on an exterior of the housing. The system can further include a glucose sensor configured to measure a signal indicative of glucose levels of a user and including one or more external electrical contacts. A pump holder can include a tray configured to releasably retain the ambulatory infusion pump and a sensor nest configured to receive the glucose sensor. The tray can be configured such that when the glucose sensor is received within the sensor nest and the ambulatory infusion pump is retained on the tray, the electrical connector on the exterior of the housing of the ambulatory infusion pump is directly contacting the one or more external electrical contacts of the glucose sensor to facilitate transfer of power from the battery of the ambulatory infusion pump to the glucose sensor.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an embodiment of an ambulatory infusion pump for use with embodiments of the disclosure.

FIG. 2 is a block diagram of the ambulatory infusion pump of FIG. 1.

FIGS. 3A-3B are an alternate embodiment of an ambulatory infusion pump for use with embodiments of the disclosure.

FIG. 4 depicts a schematic representation of an infusion pump system according to an embodiment of the disclosure.

FIG. 5 depicts an infusion pump system according to an embodiment of the disclosure.

FIG. 6 depicts the pump holder of the infusion pump system of FIG. 5.

FIGS. 7A-7C depict the CGM sensor of the infusion pump system of FIG. 5.

FIG. 8 depicts a portion of the infusion pump of the infusion pump system of FIG. 5.

FIG. 9 depicts the pump holder of the infusion pump system of FIG. 5 with the CGM sensor and an infusion cannula inserted therein.

FIGS. 10A-10B depict the infusion pump system of FIG. 5.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

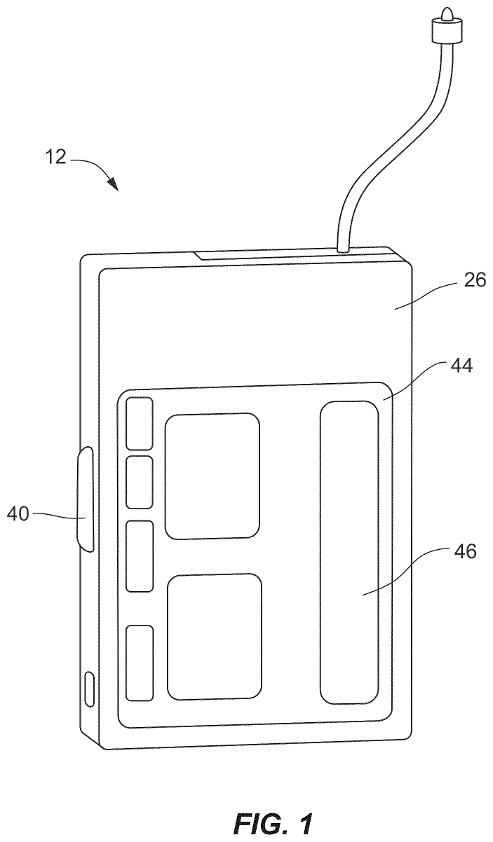

FIG. 1 depicts an exemplary medical device that can be used with embodiments of the disclosure. In this embodiment, the medical device is configured as a pump 12, such as an infusion pump, that can include a pumping or delivery mechanism and reservoir for delivering medicament to a patient and an output/display 44. The type of output/display 44 may vary as may be useful for a particular application. The output/display 44 may include an interactive and/or touch sensitive screen 46 having an input device such as, for example, a touch screen comprising a capacitive screen or a resistive screen. The pump 12 may additionally include a keyboard, microphone, or other input device known in the art for data entry, which may be separate from the display. The pump 12 may also include a capability to operatively couple to one or more blood glucose meters (BGMs) or continuous blood glucose monitors (CGMs) and/or one or more secondary devices such as a remote display, a remote control device, a laptop computer, personal computer, tablet computer, a mobile communication device such as a smartphone, a wearable electronic watch, smart ring, electronic health or fitness monitor, or personal digital assistant (PDA), a CGM display etc.

In one embodiment, the medical device can be a portable pump configured to deliver insulin to a patient. Further details regarding such pump devices can be found in U.S. Pat. No. 8,287,495, which is incorporated herein by reference in its entirety. In other embodiments, the medical device can be an infusion pump configured to deliver one or more additional or other medicaments to a patient.

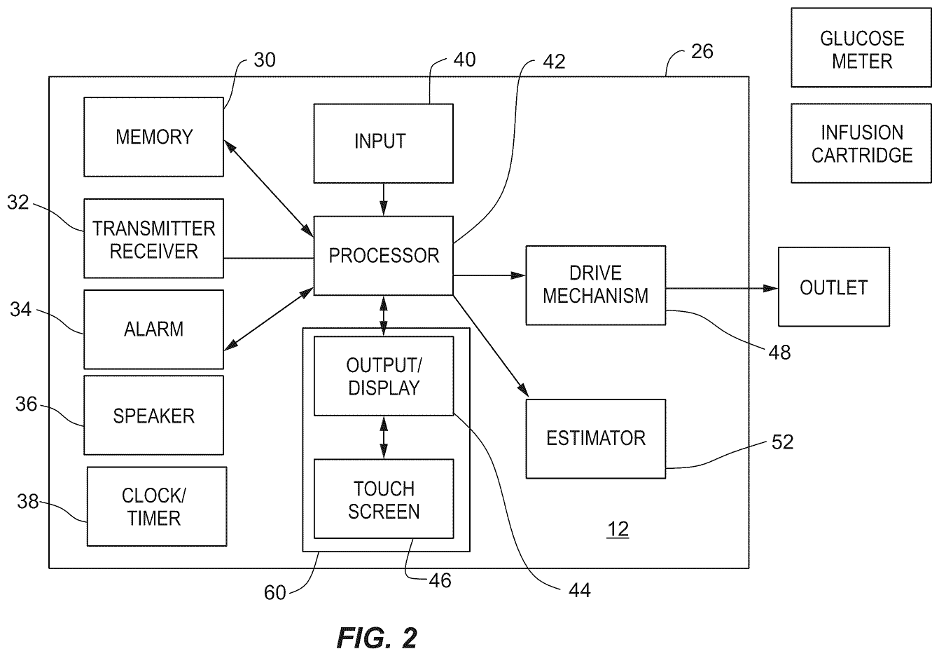

FIG. 2 illustrates a block diagram of some of the features that can be used with embodiments, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52, and a microphone (not pictured). One embodiment of a user interface as shown in FIG. 2 is a graphical user interface (GUI) 60 having a touch sensitive screen 46 with input capability. In some embodiments, the processor 42 may communicate with one or more other processors within the pump 12 and/or one or more processors of other devices, for example, a continuous glucose monitor (CGM), display device, smartphone, etc. through the transmitter/receiver. The processor 42 may also include programming that may allow the processor 42 to receive signals and/or other data from one or more input devices, such as sensors that may sense pressure, temperature and/or other parameters.

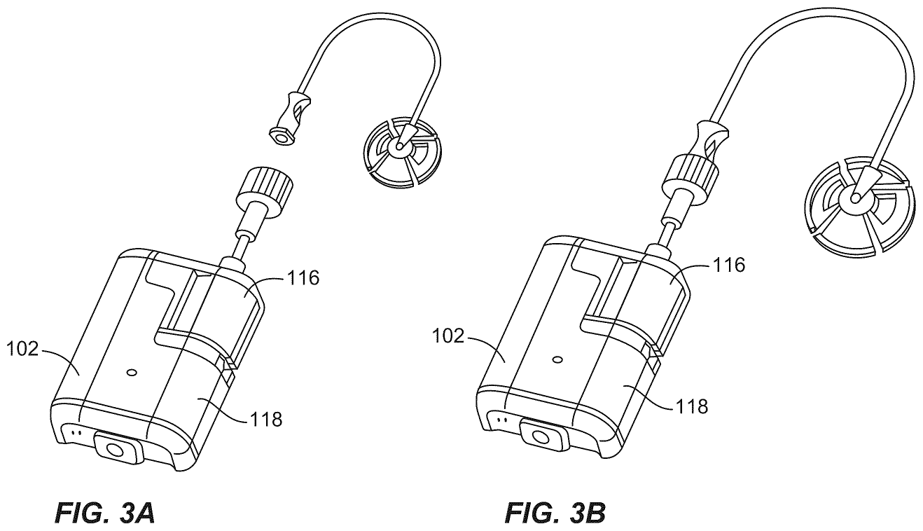

FIGS. 3A-3B depict a second infusion pump that can be used in conjunction with one or more embodiments of the ambulatory infusion pump system of the present disclosure. Pump 102 includes a pump drive unit 118 and a medicament cartridge 116. Pump 102 includes a processor 42 that may communicate with one or more processors within the pump 102 and/or one or more processors of other devices such as a remote device (e.g., a CGM device), a remote control device, or a consumer electronic device (e.g., laptop computer, personal computer, tablet computer, smartphone, electronic watch, smart ring, electronic health or fitness monitor, or personal digital assistant). The processor 42 may also include programming to receive signals and/or other data from an input device, such as, by way of example, a pressure sensor, a temperature sensor, or the like. Pump 102 also includes a processor that controls some or all of the operations of the pump. In some embodiments, pump 102 receives commands from a separate device for control of some or all of the operations of the pump. Such separate device can include, for example, a dedicated remote device or a consumer electronic device such as a smartphone having a processor executing an application configured to enable the device to transmit operating commands to the processor 42 of pump 102. In some embodiments, processor 42 can also transmit information to one or more separate devices, such as information pertaining to device parameters, alarms, reminders, pump status, etc. Such separate device can include any remote display, remote device, remote control, or a consumer electronic device as described previously.

Pump 102 can also incorporate any or all of the features described with respect to pump 12 in FIG. 2. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. Further details regarding such pumps can be found in U.S. Pat. No. 10,279,106 and U.S. Patent Publication Nos. 2016/0339172 and 2017/0049957, each of which is hereby incorporated herein by reference in its entirety.

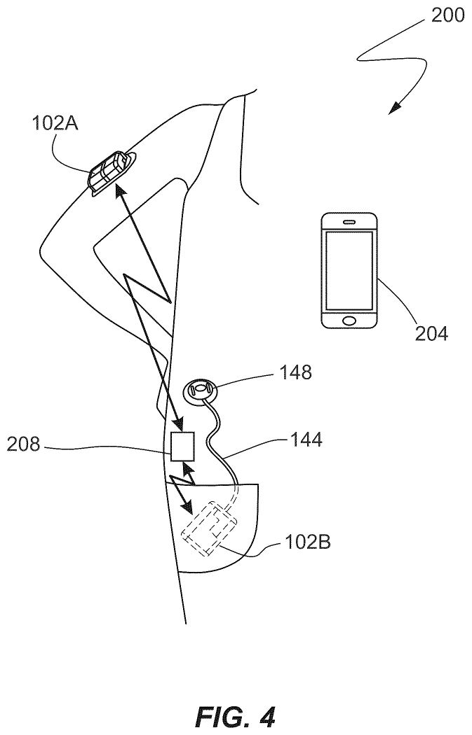

FIG. 4 depicts a schematic representation of a pump system 200 according to an embodiment of the disclosure. System 200 includes a user-wearable infusion pump such as pump 12 or pump 102 described above. In embodiments, a user can alternatively wear the pump 102A directly on the body or place the pump 102B in the user's pocket or other location near the body with infusion tubing 144 extending to an infusion set 148 on the user's body. The system 200 also includes a continuous glucose monitoring (CGM) sensor with a corresponding transmitter 208. The CGM sensor obtains measurements relating to glucose levels in the body and the transmitter can communicate that information to pump 102A/B. Pump 208 can then use the glucose data in making therapy determinations. The system can also include one or more devices such as a smartphone 204 or other multi-purpose consumer electronic device capable of operating a software application to communicate with and/or control the pump and, alternatively or additionally, a dedicated remote device designed specifically for use with pump 102A/102B. The smartphone 204 or other remote electronic device can in some embodiments also be capable of communication with CGM sensor/transmitter 208 and can calculate therapy commands for execution by the pump 102A/102B based on the glucose levels.

Although as described above infusion pumps and continuous glucose monitors are separate devices, there are disadvantages to this configuration. For example, the requirement for Bluetooth or other wireless communications between the pump and the CGM require the devices to be paired. This requires the user to undertake a complex pairing procedure to pair the two devices and errors can arise if the devices become unpaired or communication issues arise between the devices. The CGM sensor and the pump also require two different attachment sites and separate insertions into the user's body. In addition, the separate devices each require their own batteries, processors, communications devices and associated circuitry. Embodiments described herein integrate the CGM with the ambulatory infusion pump to address each of these disadvantages.

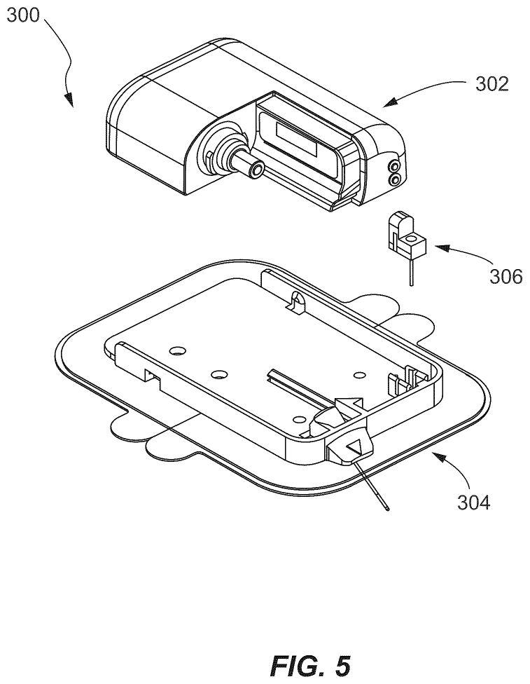

Referring to FIG. 5, an ambulatory infusion pump system 300 according to an embodiment of the disclosure is depicted. Ambulatory infusion pump system 300 is an integrated CGM and insulin pump system that can include a user-wearable infusion pump 302, a pump holder or tray 304 and a CGM sensor 306.

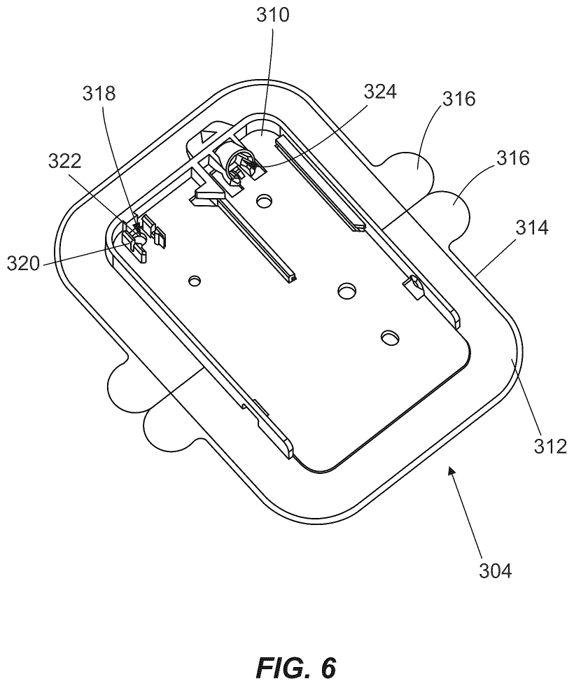

Pump holder 304 is also depicted in FIG. 6. Pump holder 304 can include a tray body 310 attached to an adhesive patch 312 that can releasably attach the pump holder 304 to a body of a user. A patch liner 314 comprising one or more pieces can include tabs 316 for removing patch liner 314 to expose the adhesive underside of adhesive patch 312 for attachment to the user. Tray body 310 can include a number of features related to guiding and releasably retaining an infusion pump on tray body 310, which are not described in detail herein. Further details regarding such features can be found in U.S. Patent Publication Nos. 2024/0050650 and 2024/0226423, each of which is hereby incorporated herein by reference. Pump holder 304 can include a sensor nest 318 defined on tray body 310 sized and shaped to receive a CGM sensor such as sensor 306. The size and shape of sensor nest 318 can vary based on the configuration of a particular sensor. In the depicted embodiment, sensor nest 318 comprises a pair of nest walls 320 that can include mechanical mating features configured to provide a snap-fit to releasably retain a CGM sensor therein. A probe aperture 322 between walls 320 enables insertion of a sensor probe of the CGM sensor through the pump holder 304 and into the user's body. Tray body 310 can also include an infusion set nest 324 configured to releasably retain a cannula assembly for delivery of medicament to the user therein.

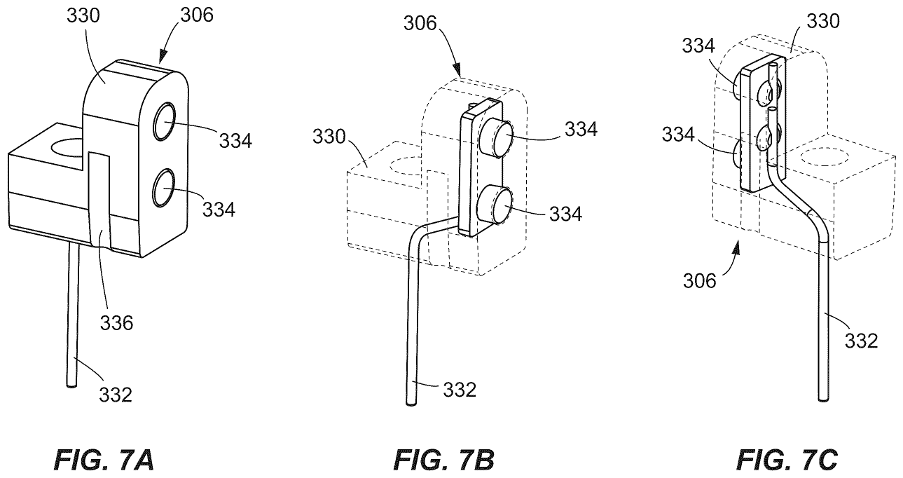

A CGM sensor 306 that can be used with a system 300 as disclosed herein is depicted in FIGS. 7A-7C. CGM sensor 306 can include a sensor body 330 with a sensor probe 332 extending from the body that is configured to be inserted into the body of the user. Sensor probe 332 can be electrically coupled within sensor body 330 to a plurality of electrical contacts 334 that can be exposed on an exterior of sensor body 330 to transmit electrical signals from the sensor probe 332. Although two electrical contacts 334 are depicted greater or fewer electrical contacts can be employed. In addition, although the electrical contacts 334 are depicted as being oriented vertically with respect to one another, other orientations are possible such as horizonal, coaxial, etc. The particular geometry and size of sensor body 330 is exemplary only and system can be adapted as needed for various other applications. In the depicted embodiment, sensor body 330 includes a retention slot 336 on each side of sensor body 330 that interface with retention features in sensor nest 318 of pump holder 304 to aid in retaining CGM sensor 304 in sensor nest 318.

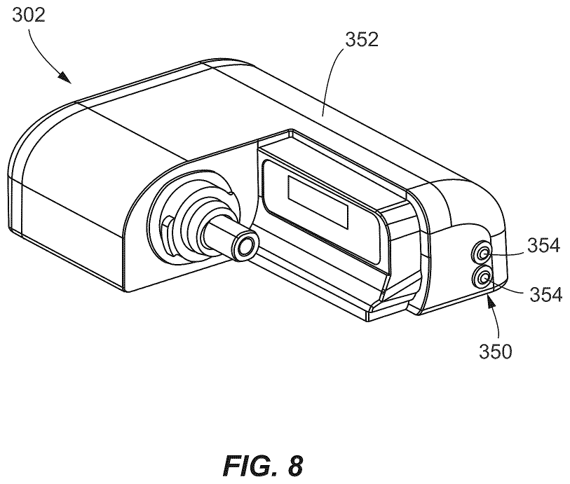

FIG. 8 depicts a portion of an ambulatory infusion pump 302 for use in an ambulatory infusion pump system 300 according to the disclosure. Ambulatory infusion pump 302 can include an electrical connector 350 on an exterior surface of a housing 352 of pump 302. In the depicted embodiment, electrical connector 350 comprises a pair of spring-loaded pogo pins 354. The pair of vertically oriented pins 354 correspond to the number and orientations of the electrical contacts 334 of CGM sensor 306 such that in embodiments in which CGM sensor has a different number, orientation, and/or configuration of electrical contacts, the electrical connector 350 on ambulatory infusion pump 302 can similarly be redesigned to interface with a given CGM sensor design. In various other embodiments, other known types of electrical connections can be employed. Additional circuitry would electrically couple the electrical connector 350 with the internal electrical components of the pump 302, such as the battery and processor.

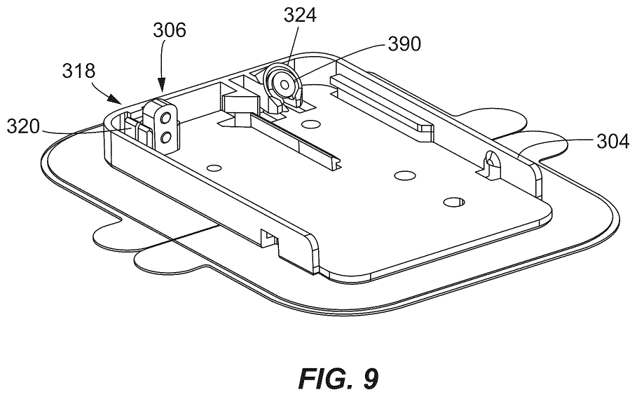

FIG. 9 depicts the pump holder 304 of ambulatory infusion pump system 300 with the CGM sensor 306 and an infusion cannula 390 inserted through pump holder 304 (and into a user's body). CGM sensor 306 nests within sensor nest 318 of tray body 310 with the mating features of nest walls 320 providing a snap-fit with retention slots 336 in CGM sensor 306 to releasably retain the CGM sensor 306 within the sensor nest 318. Infusion cannula 390 is similarly releasably retained within the infusion set nest 324.

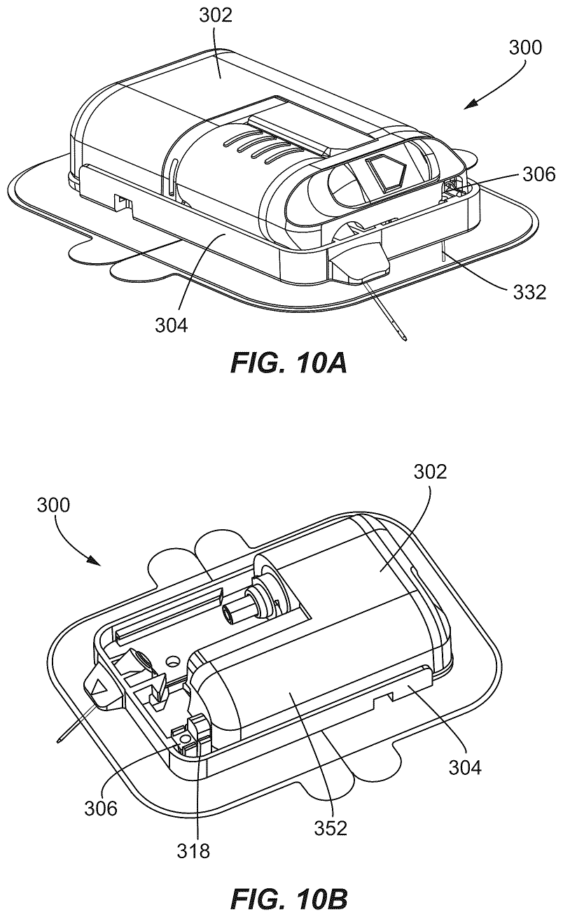

Ambulatory infusion pump 302 can then be releasably attached to the tray body 310 of pump holder 304 as shown in FIGS. 10A-10B. FIG. 10A depicts the assembled system 300 and FIG. 10B depicts the system with a cartridge of the ambulatory infusion pump 302 removed for sake of clarity. Sensor nest 318 is positioned on tray body 310 and is configured to retain CGM sensor 306 in manner such that when pump 302 is slid onto tray body 310, the pogo pins 354 (or other electrical contacts) of electrical connector 350 on pump housing 352 are brought into direct contact with the electrical contacts 334 of CGM sensor 306. The creates a direct electrical coupling from the CGM sensor 306 to the internal processor of the pump 302 via the electrical connectors 334, 350 of the CGM sensor 306 and pump 302 and internal circuitry in the pump 302.

In embodiments disclosed herein, a CGM sensor 306 is therefore powered through a direct electrical connection with an insulin pump 302 such that the CGM sensor receives power from the internal battery in the pump 302. This eliminates the need for a traditional battery within the CGM sensor, reducing its size and cost. Elimination of the battery further eliminates the need for replacing the battery in the sensor, simplifying device maintenance and enhancing reliability since there will be no gaps in sensor readings due to a dead battery.

The insulin pump 302 and CGM sensor 306 are both housed within the tray 310 of the pump holder in a manner that ensures power transfer between the pump and the sensor while maintaining a sleek, unified appearance on the body. This reduces the number of visible devices on the body, providing a more discreet and visually appealing system.

In these embodiments, communications between the CGM sensor and pump can also be accomplished via the direct electrical connection. Pump can include internal circuitry to transmit the signals measured by the CGM sensor to the pump processor. The processor of pump can include programming sufficient to receive the CGM signals and to process those signals for use of the glucose levels indicated by the signals in providing therapy with the pump. In such embodiments, no Bluetooth or other wireless communications between the devices is required because the CGM signals are sent to the pump utilizing the direct electrical connection between the CGM sensor and the pump. As such, the CGM does not require a complex pairing procedure and wireless connectivity issues preventing transmission of CGM data will not occur. There are therefore further cost savings in the system because the CGM also does not need a processor, wireless communications elements and associated circuitry. The battery life of the pump will also be enhanced due to reduced power consumption from not having continual wireless communications with the CGM, because the power consumption of providing power to the CGM is significantly lower that the power required for wireless communications with the CGM. In some embodiments, data obtained by the CGM sensor can be stored within a memory of the pump. This data could then be periodically synced to a smartphone application, the cloud or another external source to which the pump can periodically be connected. This can enhance data accessibility and historical data tracking, analysis and trends for patients and/or healthcare providers.

In embodiments, the pump will be able to determine if the connection with the CGM sensor has been disconnected because the pump will no longer be receiving data from the CGM and/or if the CGM loses power. In such circumstances, the pump can provide an alert informing the user that the CGM has become disconnected and/or has low or no power.

In some embodiments, the system can include a dual-purpose inserter (not pictured) to optionally allow for simultaneous insertion of both the CGM sensor and the insulin cannula for the pump into the body. This can simplify the insertion process for patients. Separate inserters can also be used. This may be beneficial in situations where the CGM sensor needs to be replaced more frequently than the cannula, or vice versa.

In embodiments, an ambulatory infusion pump system can include an ambulatory infusion pump including a housing, a reservoir configured to contain a medicament, a pumping mechanism configured to deliver the medicament from the reservoir to a user, a battery, and an electrical connector on an exterior of the housing. A pump holder can include a tray configured to releasably retain the ambulatory infusion pump, the pump holder including a sensor nest configured to receive a glucose sensor. The tray can be configured such that when the glucose sensor is received within the sensor nest and the ambulatory infusion pump is retained on the tray, the electrical connector on the exterior of the housing of the ambulatory infusion pump is directly contacting one or more external electrical contacts of the glucose sensor to facilitate transfer of power from the battery of the ambulatory infusion pump to the glucose sensor.

In some embodiments, the sensor nest comprises a pair of sensors walls sized to receive the glucose sensor therein.

In some embodiments, the sensor nest comprises one or more mechanical mating features configured to releasably retaining the glucose sensor therein.

In some embodiments, the one or more mechanical mating features provide a snap fit with the glucose sensor.

In some embodiments, the tray includes a probe aperture through base surface of the tray adjacent the sensor nest configured to enable a sensor probe of the glucose sensor to be inserted therethrough.

In some embodiments, the tray further includes an infusion set nest configured to releasably retain a cannula assembly.

In some embodiments, the electrical connector on the exterior of the housing of the ambulatory infusion pump comprises one or more pogo pins.

In some embodiments, a number of pogo pins corresponds to a number of electrical contacts of the glucose sensor.

In some embodiments, the glucose sensor does not include an internal battery.

In some embodiments, the contact between the electrical connector on the exterior of the housing of the ambulatory infusion pump and the one or more external electrical contacts of the glucose sensor further facilitates communications between the glucose sensor and the ambulatory infusion pump.

In embodiments, an ambulatory infusion pump system can include an ambulatory infusion pump including a housing, a reservoir configured to contain a medicament, a pumping mechanism configured to deliver the medicament from the reservoir to a user, and a battery. A pump holder can include a tray configured to releasably retain the ambulatory infusion pump, the pump holder including a sensor nest configured to receive a glucose sensor. The tray can be configured such that when the glucose sensor is received within the sensor nest and the ambulatory infusion pump is retained on the tray, the ambulatory infusion pump is directly contacting the glucose sensor to facilitate transfer of power from the battery of the ambulatory infusion pump to the glucose sensor via a direct electrical connection.

In some embodiments, the sensor nest comprises a pair of sensors walls sized to receive the glucose sensor therein.

In some embodiments, the sensor nest comprises one or more mechanical mating features configured to releasably retaining the glucose sensor therein.

In some embodiments, the one or more mechanical mating features provide a snap fit with the glucose sensor.

In some embodiments, the tray includes a probe aperture through base surface of the tray adjacent the sensor nest configured to enable a sensor probe of the glucose sensor to be inserted therethrough.

In some embodiments, the tray further includes an infusion set nest configured to releasably retain a cannula assembly.

In some embodiments, the direct electrical connection is between an electrical connector on the exterior of the housing of the ambulatory infusion pump and one or more external electrical contacts of the glucose sensor.

In some embodiments, the electrical connector on the exterior of the housing of the ambulatory infusion pump comprises a number of pogo pins corresponding to a number of electrical contacts of the glucose sensor.

In some embodiments, the glucose sensor does not include an internal battery.

In some embodiments, the direct electrical connection between the ambulatory infusion pump and the glucose sensor further facilitates communications between the glucose sensor and the ambulatory infusion pump.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425; 12,214,159; and 12,357,751 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924; 2024/0399051; 2024/408303; 2024/0416032; 2024/0416033; 2025/0099674; 2025/0099675 2025/0099678; 2025/0099679; and 2025/0108162 and commonly owned U.S. patent application Ser. Nos. 17/368,968; 17/896,492; 18/398,543; 18/962,169; 19/003,140; 19/003,164; 19/119,554; 19/134,333; 19/205,083; 19/220,426; 19/221,933; 19/225,150; and Ser. No. 19/252,256.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

1. An ambulatory infusion pump system, comprising:
an ambulatory infusion pump including a housing, a reservoir configured to contain a medicament, a pumping mechanism configured to deliver the medicament from the reservoir to a user, a battery, and an electrical connector on an exterior of the housing; and
a pump holder including a tray configured to releasably retain the ambulatory infusion pump, the pump holder including a sensor nest configured to receive a glucose sensor, wherein the tray is configured such that when the glucose sensor is received within the sensor nest and the ambulatory infusion pump is retained on the tray, the electrical connector on the exterior of the housing of the ambulatory infusion pump is directly contacting one or more external electrical contacts of the glucose sensor to facilitate transfer of power from the battery of the ambulatory infusion pump to the glucose sensor.

2. The ambulatory infusion pump system of claim 1, wherein the sensor nest comprises a pair of sensors walls sized to receive the glucose sensor therein.

3. The ambulatory infusion pump system of claim 1, wherein the sensor nest comprises one or more mechanical mating features configured to releasably retaining the glucose sensor therein.

4. The ambulatory infusion pump system of claim 3, wherein the one or more mechanical mating features provide a snap fit with the glucose sensor.

5. The ambulatory infusion pump system of claim 1, wherein the tray includes a probe aperture through base surface of the tray adjacent the sensor nest configured to enable a sensor probe of the glucose sensor to be inserted therethrough.

6. The ambulatory infusion pump system of claim 1, wherein the tray further includes an infusion set nest configured to releasably retain a cannula assembly.

7. The ambulatory infusion pump system of claim 1, wherein the electrical connector on the exterior of the housing of the ambulatory infusion pump comprises one or more pogo pins.

8. The ambulatory infusion pump system of claim 7, wherein a number of pogo pins corresponds to a number of electrical contacts of the glucose sensor.

9. The ambulatory infusion pump system of claim 1, wherein the glucose sensor does not include an internal battery.

10. The ambulatory infusion pump system of claim 1, wherein the contact between the electrical connector on the exterior of the housing of the ambulatory infusion pump and the one or more external electrical contacts of the glucose sensor further facilitates communications between the glucose sensor and the ambulatory infusion pump.

11. An ambulatory infusion pump system, comprising:
an ambulatory infusion pump including a housing, a reservoir configured to contain a medicament, a pumping mechanism configured to deliver the medicament from the reservoir to a user, and a battery; and
a pump holder including a tray configured to releasably retain the ambulatory infusion pump, the pump holder including a sensor nest configured to receive a glucose sensor, wherein the tray is configured such that when the glucose sensor is received within the sensor nest and the ambulatory infusion pump is retained on the tray, the ambulatory infusion pump is directly contacting the glucose sensor to facilitate transfer of power from the battery of the ambulatory infusion pump to the glucose sensor via a direct electrical connection.

12. The ambulatory infusion pump system of claim 11, wherein the sensor nest comprises a pair of sensors walls sized to receive the glucose sensor therein.

13. The ambulatory infusion pump system of claim 11, wherein the sensor nest comprises one or more mechanical mating features configured to releasably retaining the glucose sensor therein.

14. The ambulatory infusion pump system of claim 13, wherein the one or more mechanical mating features provide a snap fit with the glucose sensor.

15. The ambulatory infusion pump system of claim 11, wherein the tray includes a probe aperture through base surface of the tray adjacent the sensor nest configured to enable a sensor probe of the glucose sensor to be inserted therethrough.

16. The ambulatory infusion pump system of claim 11, wherein the tray further includes an infusion set nest configured to releasably retain a cannula assembly.

17. The ambulatory infusion pump system of claim 11, wherein the direct electrical connection is between an electrical connector on the exterior of the housing of the ambulatory infusion pump and one or more external electrical contacts of the glucose sensor.

18. The ambulatory infusion pump system of claim 17, wherein the electrical connector on the exterior of the housing of the ambulatory infusion pump comprises a number of pogo pins corresponding to a number of electrical contacts of the glucose sensor.

19. The ambulatory infusion pump system of claim 11, wherein the glucose sensor does not include an internal battery.

20. The ambulatory infusion pump system of claim 11, the direct electrical connection between the ambulatory infusion pump and the glucose sensor further facilitates communications between the glucose sensor and the ambulatory infusion pump.
